(19) United States
(12) Patent Application Publication
(10) Pub. No.: US 2018/0153800 A1
(43) Pub. Date: Jun. 7, 2018

(54) PERSONAL LUBRICANT PROVIDING SEQUENTIAL MULTISENSORIAL PERCEPTIONS AND ARTICLES INCORPORATING SUCH LUBRICANT

(71) Applicant: Church & Dwight Co., Inc., Princeton, NJ (US)

(72) Inventors: Elisabeth Memin, North Plainfield, NJ (US); Albert Nazareth, Mercerville, NJ (US); Kirsten Cluver, Hamilton, NJ (US); Caihua Wang, Kendall Park, NJ (US); Melis Cakirer, Denville, NJ (US); Jason Kieke, Freehold, NJ (US)

(73) Assignee: Church & Dwight Co. Inc., Princeton, NJ (US)

(21) Appl. No.: 15/833,127

(22) Filed: Dec. 6, 2017

Related U.S. Application Data
(60) Provisional application No. 62/430,688, filed on Dec. 6, 2016.

(51) Int. Cl. A61K 9/00; A61F 6/04

(52) U.S. Cl. CPC A61K 9/0034; A61F 6/04; A61K 9/0014

(57) ABSTRACT

The present disclosure provides lubricant compositions suitable for use on human tissue and effective to provide multiple sensations sequentially and/or in varying intensities. The lubricants can include a lubricating base and at least two different sensorial agents effective to impart different sensations, such as warming, cooling, tingling, refreshing, and numbing. The lubricants particularly can be suitable for use in sexual experiences so that the imparted sensations can increase and/or improve such experiences. The present disclosure further provides condoms incorporating such lubricant compositions.

### CROSS-REFERENCE TO RELATED APPLICATIONS

The present application claims priority to U.S. Provisional Patent Application No. 62/430,688, filed Dec. 6, 2016, the disclosure of which is incorporated here by reference.

### FIELD OF THE DISCLOSURE

The present disclosure relates to compositions for application to human tissue and articles incorporating such compositions. More particularly, the disclosure relates to lubricant compositions that provide lubricating effects and various sensory perceptions to the tissue of application, as well as articles that include the lubricant composition and that are configured for contacting human tissue.

### BACKGROUND

Personal lubricants are known compositions, typically in the form of a gel, cream, or lotion, that are intended for use in intimate contact in various sexual activities. It has recently become increasingly popular to add various functional ingredients to personal lubricant —i.e., ingredients providing effects beyond lubrication. Sensorial agents effective to provide sensations such as warming, cooling, and tingling can provide benefits for men and women to enhance pleasurable feelings during sexual activity and/or to heighten sexual arousal.

Personal lubricants comprising such functional ingredients are typically designed to cause physiological and/or physical changes in the area of the tissue to which they are applied. The functional ingredients can exhibit a number of different modes of activity. For examples, certain materials are known to be self-warming when exposed to moisture, other materials are known to act on nerve endings to simulate a perceived sensation, and further materials are known to affect local blood flow in the tissue. U.S. Patent Publication No. 2015/0250704 to Romanoschi et al. discloses lubricant compositions comprising at least one sensorial agent in a silicone fluid carrier. Although personal lubricants including functional ingredients such as sensorial agents are known, there remains a desire in the field to provide further lubricants that can stimulate a variety of sensations in and around the tissue to which they are applied and thus further enhance the pleasurable feelings noted above.

Personal lubricants are commonly applied to articles that are configured for application to human tissue and particularly to articles, such as condoms, which have been marketed with various shapes, textures, and lubricant compositions applied thereto. Lubricants for combination with a condom may be packaged separately as personal lubricants, while others are applied to condoms prior to packaging. Some lubricants for combination with condoms have particular physiological effects. For example, U.S. Pat. No. 4,840,188 to Heidenfelder discloses the coating of the interior surface of a condom with a local anesthetic such as benzocaine to provide desensitization. Thus, while articles including lubricants providing physiological effects are known, there remains a desire in the field to provide further articles including lubricants that can stimulate a variety of sensations in and around the tissue to which the articles are applied.

### SUMMARY OF THE DISCLOSURE

The present disclosure provides lubricant compositions that provide lubricating qualities as well as multiple sensations, such as warming, cooling, tingling, refreshing, and numbing. The lubricant compositions particularly are effective to provide two or more sensations upon application to human tissue, preferably at least three sensations. The lubricant compositions include sensorial agents that are effective to impart different sensations in a sequential and temporally separated fashion. The provided sensations can have varying intensities and can be time-released. The different sensations can have defined, easily recognizable durations with distinctive onset times and distinctive changes between sensations. The lubricants provide enjoyable and pleasurable sequences of sensations to be felt while providing safe and effective lubrication. The lubricant provides multiple perceivable sensations with the sensation intensity changing over time during use.

The present disclosure also provides articles, such as condoms, that are configured for application to human tissue and that includes lubricant compositions as described herein. The lubricant compositions are preferably applied to the condoms prior to packaging so that the lubricant is present on one or more surfaces of the condom at the time of unpackaging the condom.

In one or more embodiments, a lubricant according to the present disclosure can comprise the following: a lubricating base; a first sensorial agent effective to impart a first sensation to contacted human tissue; and a second sensorial agent effective to impart a second sensation to the contacted human tissue, the second sensation being different from the first sensation. In particular, the lubricant can be configured such that onset of the first sensation and onset of the second sensation are sequential and are temporally separated. In further embodiments, the lubricant can be defined in relation to one or more of the following statements, which can be combined in any number and order.

The lubricant further can comprise a third sensorial agent effective to impart a third sensation to the contacted human tissue, the third sensation being different from the first sensation and the second sensation, wherein onset of the third sensation and onset of the second sensation are sequential and temporally separated.

The intensity of one or both of the first sensation and the second sensation can vary over the time of contact with the human tissue.

The intensity of one or both of the first sensation and the second sensation can gradually increase to a maximum intensity and then gradually decreases.

The onset of the first sensation and onset of the second sensation can be temporally separated by a time of about 5 seconds or greater.

The onset of the first sensation and onset of the second sensation can be temporally separated by a time of about 5 seconds to about 5 minutes.

The onset of the second sensation and the onset of the third sensation can be temporally separated by a time of about 5 seconds or greater.

The onset of the second sensation and onset of the third sensation can be temporally separated by a time of about 5 seconds to about 5 minutes.

The first sensation and the second sensation can be each independently imparted for a duration of about 30 seconds to about 15 minutes.

The third sensation can be independently imparted for a duration of about 30 seconds to about 15 minutes.

The first sensation can substantially cease before the onset of the second sensation.

The second sensation can substantially cease before the onset of the third sensation.

The imparting of the first sensation can overlap with the imparting of the second sensation by a time of no more than 3 minutes or no more than 2 minutes.

The imparting of the second sensation can overlap with the imparting of the third sensation by a time of no more than 3 minutes or no more than 2 minutes.

The first sensation and the second sensation can be independently selected from the group consisting of warming, cooling, tingling, numbing, and refreshing.

The third sensation can be selected from the group consisting of warming, cooling, tingling, numbing, and refreshing.

One of the first sensorial agent and the second sensorial agent can be a warming sensorial agent, and the other of the first sensorial agent and the second sensorial agent can be a cooling sensorial agent.

One of the first sensorial agent, the second sensorial agent, and the third sensorial agent can be a warming sensorial agent, and another of the first sensorial agent, the second sensorial agent, and the third sensorial agent can be a cooling sensorial agent, and the remaining of the first sensorial agent, the second sensorial agent, and the third sensorial agent can be a tingling sensorial agent.

The onset of the first sensation can occur within 30 seconds of initial contact to the human skin, and the onset of the second sensation can be delayed for at least 1 minute after initial contact to the human skin.

In some embodiments, the present disclosure further can be related to methods of providing lubrication and varying sensations to human tissue. Such methods can comprise applying a lubricant as described herein to human tissue. The methods particularly can relate to use of the lubricant compositions during a sexual activity, including sexual intercourse.

In some embodiments, the present disclosure further can be related to an article. In particular, the present disclosure can provide a packaged condom. Such packaged condom can comprise a male genital engaging tubular sheath having an inner surface and an outer surface, and the packaged condom can further comprise a lubricant applied to at least a portion of one or both of the inner surface and the outer surface of the tubular sheath. In particular, the lubricant can have any composition as otherwise described herein. In further embodiments, the packaged condom can be defined in relation to one or more of the following statements, which can be combined in any number and order.

The lubricant applied to one or both of the inner surface and the outer surface of the tubular sheath can comprise the following: a lubricating base; a first sensorial agent effective to impart a first sensation to contacted human tissue; and a second sensorial agent effective to impart a second sensation to the contacted human tissue, the second sensation being different from the first sensation. In particular, the lubricant can be configured such that onset of the first sensation and onset of the second sensation are sequential and are temporally separated.

The lubricant applied to one or both of the inner surface and the outer surface of the tubular sheath further can comprise a third sensorial agent effective to impart a third sensation to the contacted human tissue, the third sensation being different from the first sensation and the second sensation, wherein onset of the third sensation and onset of the second sensation are sequential and temporally separated.

The intensity of one or both of the first sensation and the second sensation can vary over the time of contact with the human tissue.

The intensity of one or both of the first sensation and the second sensation can gradually increase to a maximum intensity and then gradually decreases.

The onset of the first sensation and onset of the second sensation can be temporally separated by a time of about 5 seconds or greater.

The onset of the first sensation and onset of the second sensation can be temporally separated by a time of about 5 seconds to about 5 minutes.

The onset of the second sensation and the onset of the third sensation can be temporally separated by a time of about 5 seconds or greater.

The onset of the second sensation and onset of the third sensation can be temporally separated by a time of about 5 seconds to about 5 minutes.

The first sensation and the second sensation can be each independently imparted for a duration of about 30 seconds to about 15 minutes.

The third sensation can be independently imparted for a duration of about 30 seconds to about 15 minutes.

The first sensation can substantially cease before the onset of the second sensation.

The second sensation can substantially cease before the onset of the third sensation.

The imparting of the first sensation can overlap with the imparting of the second sensation by a time of no more than 3 minutes or no more than 2 minutes.

The imparting of the second sensation can overlap with the imparting of the third sensation by a time of no more than 3 minutes or no more than 2 minutes.

The first sensation and the second sensation can be independently selected from the group consisting of warming, cooling, tingling, numbing, and refreshing.

The third sensation can be selected from the group consisting of warming, cooling, tingling, numbing, and refreshing.

One of the first sensorial agent and the second sensorial agent can be a warming sensorial agent, and the other of the first sensorial agent and the second sensorial agent can be a cooling sensorial agent.

One of the first sensorial agent, the second sensorial agent, and the third sensorial agent can be a warming sensorial agent, and another of the first sensorial agent, the second sensorial agent, and the third sensorial agent can be a cooling sensorial agent, and the remaining of the first sensorial agent, the second sensorial agent, and the third sensorial agent can be a tingling sensorial agent.

The onset of the first sensation can occur within 30 seconds of initial contact to the human skin, and the onset of the second sensation can be delayed for at least 1 minute after initial contact to the human skin.

The same lubricant composition may be applied to the inner surface and the outer surface of the tubular sheath.

A first lubricant composition may be applied to the inner surface of the tubular sheath, and a second, different lubricant composition may be applied to the outer surface of the tubular sheath.

The amount of the lubricant composition applied to the inner surface and the outer surface of the tubular sheath can the same or can be different.

One of the inner surface and the outer surface of the tubular sheath can include a the presently described lubricant, and the other of the inner surface and the outer surface of the tubular sheath can include a known lubricant composition.

### BRIEF DESCRIPTION OF THE FIGURES

FIG. 1 is an illustration showing a partial, perspective view of a condom according to an embodiment of the present disclosure;

FIG. 2 is a chart showing the temporal sensation profile of a lubricant composition in a mouth and lips test;

FIG. 3 is a chart showing the temporal sensation profile of a lubricant composition in a bedroom panel;

FIG. 4 is a chart showing intensity rating during the temporal sensation profile described in relation to FIG. 3;

FIG. 5 is a chart showing the temporal sensation profile of a lubricant composition in a mouth and lips test, including the intensity rating; and

FIG. 6 is a chart showing the temporal sensation profile: of a lubricant composition in a mouth and lips test, including the intensity rating.

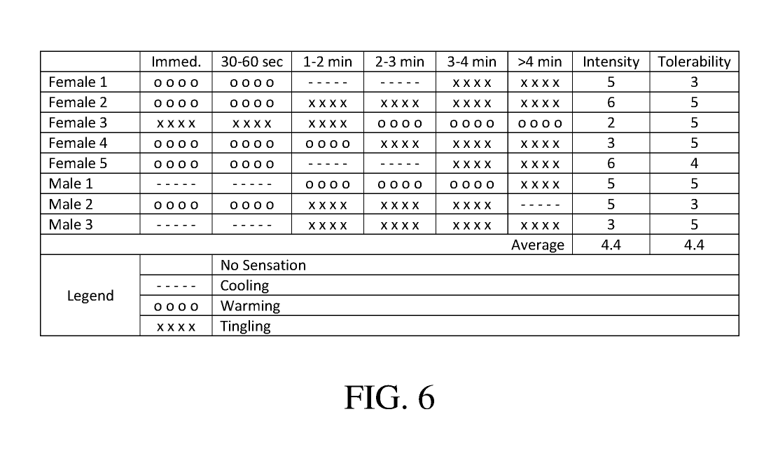

### DETAILED DESCRIPTION OF THE DISCLOSURE

The invention now will be described more fully hereinafter through reference to various embodiments. These embodiments are provided so that this disclosure will be thorough and complete, and will fully convey the scope of the invention to those skilled in the art. Indeed, the invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

The present disclosure relates to lubricants—i.e., compositions that provide lubrication to the tissue to which they are applied. Personal lubricants in particular are known to be compositions adapted to reduce friction and/or increase comfort during sexual activities. Human tissue to which the lubricants are intended for application can include the skin and mucosa and can particularly include male and/or female genitalia. The lubricants are non-toxic and are substantially or completely non-irritating to the tissue of application.

A lubricant according to embodiments of the present disclosure can comprise a lubricating base and two or more sensorial agents. A lubricating base can be any material that is understood to provide a lubricating effect that is safe for use on human tissue. The lubricating base can comprise a water-based material, a silicone-based material, a glycol, and/or an oil. In preferred embodiments, a silicone-based material, such as a dimethicone material can be used as the lubricating base. Further, non-limiting examples of materials that can be used in the lubricating base include isododecane, dimethicone/vinyl dimethicone crosspolymer, polyethylene glycol, propylene glycol, glyceryl polymethacrylate, carboxymethyl cellulose, and mineral oil. It is understood that the lubricating base can comprise a single material or be a mixture of materials. In particular, any one material listed herein or any two, three, or more materials listed herein may be combined to form the lubricating base. The lubricating base can comprise about 50% to about 99.9% by weight of the total lubricant composition, particularly about 75% to about 99.8%, about 90% to about 99.7%, or about 95% to about 99.6% by weight of the total lubricant composition. In some embodiments, the lubricating base can comprise at least 50%, at least 75%, at least 90%, or at least 95% by weight of the lubricant composition, up to a maximum content of 99.5% or 99.9% by weight, based on the total weight of the lubricant composition.

In combination with the lubricating base, a lubricant composition according to the present disclosure can also comprise two or more sensorial agents. A sensorial agent according to the present disclosure refers to a compound or mixture of compounds effective to impart at least one distinct sensation to human tissue to which it is applied. Sensorial agents as used herein preferably impart sensations including warming, cooling, tingling, numbing, and refreshing. A single sensorial agent may be effective to provide a single, distinct sensation without any substantial presence of a further sensation—e.g., only warming, or only cooling. A single sensorial agent, however, may provide a plurality of the noted sensations. When a single sensorial agent provides a plurality of sensations, it is preferable for the sensorial agent to provide a single, distinct sensation for a period of time before providing a different, distinct sensation for a period of time. If desired, useful sensorial agents may also provide overlapping sensations—e.g., providing warming and cooling at the same time. As further noted below, such overlap may occur for only a defined period of time. The presence of two or more different sensorial agents in the lubricant composition thus allows for provision of two or more separate and distinct sensations that can proceed by the same or different mode of action. While it is previously been known to prepare compositions including materials that can provide multiple sensations, such compositions are limited to simultaneous provision of the multiple sensations. For example, it is known to simultaneously provide a warming sensation and a cooling sensation. The present lubricant compositions, however, are configured to provide multiple sensations separately and sequentially. The sensations likewise may be provided with varying intensities. The total concentration of sensorial agents present in the lubricant composition can be about 0.01% to about 5% by weight, about 0.05% to about 4% by weight, about 0.1% to about 3% by weight, or about 0.2% to about 2.5% by weight, based on the total weight of the lubricant composition. The combined sensorial agents can be referred to as a “sensorial agent package” in that the sensorial agents can be provided in a pre-mixture for combination with the lubricating base to form the final lubricant composition.

Sensorial agents suitable for use in the presently disclosed lubricant compositions can include a variety of materials known in the art. For example, various sensorial agents are described in U.S. Pat. Pub. No. 2007/0020221, U.S. Pat. No. 8,143,139, U.S. Pat. No. 8,084,050, and U.S. Pat. No. 6,780,443, the disclosures of which are incorporated herein by reference.

In some embodiments, a sensorial agent for use particularly provides a warming sensation. For example, a sensorial agent can be a self-warming material such as materials that generate heat and thereby impart a warming sensation when placed in contact with moisture, such as is present in human tissue. Polyethylene glycol is an example of a material whereby a warming sensation can be imparted because of the heat of dilution—i.e., energy released in the form of heat upon dilution of the material in water. As a further example, a sensorial agent can be effective to stimulate a perceived sensation of warming. Compounds effective to bind to certain groups of transient receptor potential (TRP) channels on sensory neurons can impart a warming sensation. Vanillyl butyl ether (VBE) is a specific compound that binds the TRP type VI channel (Vanilloid receptor-1 or TRPV1). Activation of thermoreceptors in this manner can generate a brief depolarization that results in a nerve impulse that is conveyed to the brain and can, in relation to TRPV-1, elicit a warming sensation at the tissue where the activation occurs. Any material effective to activate a thermosensitive ion channel (e.g., TRPV1, TRPV2, TRPV3, TRPV4, TRPM8, or TRPA1) can be suitable for use as a sensorial agent according to the present disclosure.

Non-limiting examples of sensorial agents effective for imparting a warming sensation when contacted with human tissue include the following: ethyl alcohol; niacin; jambu; nicotinic acid; zingerone; vanillyl alcohol n-butyl ether; vanillyl alcohol n-propyl ether; vanillyl alcohol isopropyl ether; vanillyl alcohol isobutyl ether; vanillyl alcohol n-amino ether; vanillyl alcohol isoamyl ether; vanillyl alcohol n-hexyl ether; vanillyl alcohol methyl ether; vanillyl alcohol ethyl ether; 4-(1-menthoxymethyl)-2-phenyl-1,3-dioxolan; 4-(1-menthoxymethyl)-2-(3′,4′-dihydroxyphenyl)-1,3-dioxolan; 4-(1-menthoxymethyl)-2-(2′-hydroxy-3′-methoxyphenyl)-1,3-dioxolan; 4-(1-menthoxymethyl)-2-(4′-methoxyphenyl)-1,3-dioxolan; 4-(1-menthoxymethyl)-2-(3′,4′-methylenedioxyphenyl)-1,3-dioxolan; 4-(1-menthoxymethyl)-2-(3′-methoxy-4′-hydroxyphenyl)-1,3-dioxolan; gingerol; methyl salicylate; shogaol; paradol; zingerone; capsaicin; dihydrocapsaicin; nordihydrocapsaicin; homocapsaicin; homodihydrocapsaicin; ethanol; tincture capsicum; eucalyptus oil; eucalyptol; capsaicin; cinnamic aldehyde; chloroform; ether; iso-Amyl alcohol; benzyl alcohol; allyl isothiocyanate; ethyl acetate; glycerin; limonene; menthol; 4-hydroxy-4-methyl-cyclohexen-2-one-1; hydro-alcohol extracts; essential oils; oleoresins, concretes or distillates of mustard seed, ginger, horseradish, curry, chilies, jalapeno, pepper, capsicum, clove, cassia, and polyethylene glycol. Further warming sensorial agents include materials with the following designations: TK 1000; Hotact® TK 1 MM; Hotact® VBE; Hotact® VEE; Heatenol™; Optaheat; Sensehot™; Isobutavan; Ethanol; Glycerin; Nonivamide; and piperine.

In some embodiments, a sensorial agent for use particularly provides a cooling sensation. For example, a sensorial agent can be effective to stimulate a perceived sensation of cooling by binding to certain receptors, such as TRP melastatin 8 (TRPM8). Menthol, for example, binds TRPM8 and also binds cold and menthol receptor 1 (CMR1). As with warming sensorial agents, receptor activation generates a brief depolarization that results in a nerve impulse to elicit a cooling sensation at the tissue where the activation occurs. Non-limiting examples of sensorial agents effective for imparting a cooling sensation when contacted with human tissue include the following: [N-(4-cyanomethylphenyl) p-menthanecarboxamide]; 2-isopropyl-5-methylcyclohexyl-4-(dimethylamino)-oxobutanoate; monomenthyl succinate (MMS); menthol ethylene glycol carbonate (MGC); menthol propylene glycol carbonate (MPC); menthone glycerol ketal; (-)-menthyl lactate; 3-(1-menthoxy) propane-1,2-diol (MPD), Coolact® agent 10, or TK-10); 3-(1-menthoxy)-2-methylpropane-1,2-diol; 3-(1-menthoxy)ethanol (Coolact® 5); 3-(1-menthoxy)propan-1-ol; 3-(1-menthoxy)butan-1-ol; (-)-isopulegol (Coolact® P); p-menthane-3,8-diol (Coolact® 38D, PMD38); (-)-monomenthyl glutarate (physcool 2, MMG); N-ethyl-p-menthane-3-carboxamide (WS3); Ethyl 3-(p-menthane-3-carboxamido)acetate (WS5); (1R,2S,5R)-N-(4-Methoxyphenyl)-p-menthanecarboxamide (WS12); 2-isopropyl-N,2,3-trimethylbutyramide (WS23); [N-([ethoxycarbonyl]methyl)-p-menthane-3-carboxamide] (WS14); N,N-dimethyl menthyl succinamide; N-benzo[1,3] dioxol-5-yl-3-p-menthanecarboxamide; N-benzooxazol-4-yl-3-p-menthanecarboxamide; Coolact® 20; and cis & trans p-Menthane-3,8-diol (PM38D, Coolact® 38D); menthyl lactate; camphor; peppermint oil; spearmint oil; eucalyptus oil; and menthyl salicylate.

In some embodiments, a sensorial agent for use particularly provides a tingling sensation. Spilanthol is an example of sensorial agents effective for imparting a tingling sensation when contacted with human tissue. It is understood, however, that one or more of the materials described above in relation to warming sensorial agents and cooling sensorial agents can be effective as a tingling sensorial agent depending upon the concentration level of the sensorial agent in the lubricant composition. In one or more embodiments, a tingling sensation can be obtained by exciting one or more cold receptors (e.g., TRPM8) or one or more heat receptors (e.g., TRPV1). As an example, capsaicin can generate tingling sensation at certain concentrations. As a further example, szechuan pepper can be effective to provide a tingling sensation that is particularly long lasting (i.e., over 15 minutes). As another example, alkyldienamide has been reported to have cooling/tingling effect while cinnamaldehyde has been reported to have a burning/tingling sensation. It is understood that the sensation provided by a sensorial agent also may be affected by the environment in which the sensorial agent is used, such as the moisture level, pH, temperature, and the presence of other compounds. Thus, a sensorial agent under a first set of conditions can provide a first sensation and, under a second set of conditions, can provide a second, different sensation.

Within each category of sensorial agents, there is a variety of compounds that can offer different intensity levels. For example, in relation to cooling sensorial agents, the cooling sensation can be assessed based on a reference. As such, the sensation imparted by [N-(4-cyanomethylphenyl) p-menthanecarboxamide] is considered to be 10 times cooler than the sensation imparted by menthol. In one or more embodiments, sensorial agents for use in the present lubricant compositions can be chosen specifically in relation to their relative warming sensation and/or their relative cooling sensation.

If desired, further components may be included in the lubricant composition in addition to the sensorial agent package and the lubricating base. For example, any material typically recognized as being useful in a personal lubricant can be included. In some embodiments, the lubricant composition can expressly exclude further ingredients. As such, a lubricant composition as described herein can consist essentially of, or consist of one or more lubricating bases and a sensorial agent package, which itself can include a plurality of sensorial agents. It is thus understood that, in one or more embodiments, the present lubricant composition can expressly exclude any material that is not a lubricating base or that is not a sensorial agent. Moreover, any specific compound disclosed herein as suitable for use can be expressly excluded from certain embodiments where it is deemed more preferable to use one or more of the further compounds otherwise disclosed herein.

The present lubricant compositions can be defined in relation to the onset of action of the multiple sensorial agents. In particular, onset of individual sensations imparted by the individual sensorial agents is sequential and is temporally separated. This contrasts with known compositions wherein multiple sensory perceptions are provided only substantially simultaneously. It is thus understood that a sequential onset of two different sensations indicates different onset times—i.e., onset of a first sensation occurs before onset of a second sensation occurs.

In the present compositions a first sensorial agent can be effective to impart a first sensation, and the composition can be configured such that the onset of the first sensation occurs substantially immediately upon contact with the tissue (i.e., within a time of less than 10 seconds or less than 5 seconds). Alternatively, a rapid onset of the first sensation can occur within a time of less than 2 minutes or less than 1 minute after contacting the tissue (e.g., including a minimum onset time of 1 second or 2 seconds). In addition, a second senstate can be effective to impart a second sensation, and the composition can be configured such that the onset of the second sensation is delayed relative to the onset of the first sensation. As such, the onset of the first sensation and onset of the second sensation are temporally separated by a substantial length of time. The delay of the onset of the second sensation relative to the onset of the first sensation preferably is about 5 seconds or greater, about 10 seconds or greater, about 15 seconds or greater, about 30 seconds or greater, or about 1 minute or greater (e.g., with a maximum temporal separation of 10 minutes in some embodiments). In some embodiments, the onset of the first sensation and onset of the second sensation are temporally separated by a time of about 5 seconds to about 10 minutes, about 5 seconds to about 5 minutes, about 15 seconds to about 10 minutes, about 15 seconds to about 5 minutes, about 30 seconds to about 5 minutes, or about 1 minute to about 5 minutes. In particular embodiments, the temporal separation can be about 5 seconds to about 5 minutes, about 20 seconds to about 2 minutes, or about 30 seconds to about 1 minute). It is understood that the foregoing also applies to the sequential and temporally separated onset of action of any further sensorial agents that may be included in the lubricant composition.

In some embodiments, the separate sensations imparted by the separate sensorial agents may overlap for a short period of time. For example, a second sensation may begin in close proximity to the time when the first sensation has substantially ceased. As such, the overlap of sensations can be about 5 seconds to about 3 minutes, about 10 seconds to about 2 minute, or about 15 seconds to about 1 minute. Preferably, two sensations overlap by no more than 2 minutes or no more than 1 minute. In some embodiment, the first sensation may substantially cease before the onset of the second sensation. As such, the onset of the second sensation may not occur until at least 5 seconds, at least 10 seconds, at least 15 seconds, at least 30 seconds, or at least 1 minute after the first sensation has substantially ceased. The temporal separation of the occurrence of separate sensations can particularly be about 5 seconds to about 5 minutes, about 10 seconds to about 2 minutes, or about 15 seconds to about 1 minute. In some embodiments, the onset of action of the second sensation may significantly or completely mask any residual sensation from the first sensorial agent. This can relate to intensity of the sensation. In particular, the intensity of the first sensation may be relatively low at the time of onset of the second sensation, and the relatively greater intensity of the second sensation can be sufficient to substantially or completely mask the much lower intensity first sensation.

The individual sensations provided by the separate sensorial agents can differ and vary in intensity. Intensity of a sensation may be evaluated on a scale. For purposes of example, a scale of zero to five may be used. In such example, a score of 0 can indicate the complete absence of the sensation, a score of 1 can indicate a very minimal feeling of the sensation, a score of 5 can indicate a strength of sensation that indicates extreme discomfort, and scores 2, 3, or 4 indicate increasing levels of strength of sensation. It is typically desirable to avoid providing an intensity at the top of the scale. Nevertheless, it can be desirable for various sensations to be provided at significantly high levels, such as up to the range of 3 or 4 on the exemplary scale. Moreover, it is understood that sensation intensity can vary between individuals. The present lubricant compositions thus can be formulated so that different sensorial agents can provide their respective sensations at substantially the same level of intensity or at different levels of intensity. For example, a low intensity warming sensation may be sequentially followed by a higher intensity cooling sensation. Likewise, a relatively high intensity warming sensation may be sequentially followed by a lower intensity cooling sensation. Further, a tingling sensation may be provided at an intensity level that is the same as, higher, or lower than either of the warming sensation and the cooling sensation.

Additionally, the three sensations may be provided in any combination of orders. A broader rating scale (e.g., 0-10) can be utilized as appropriate to provide for a wider range of classification of sensation intensity.

In one or more embodiments, the present lubricant composition can be configured such that the intensity of one or more sensations can vary over the time of contact with the human tissue. For example, a sensation may onset at a low intensity (e.g., 1 on the exemplary scale) and then rapidly or gradually increase to a maximum intensity that is in the higher range of the exemplary scale. Any maximum intensity level can be utilized. The maximum intensity may be maintained for a short time or an extended period of time. Thereafter, the intensity may rapidly or gradually decrease until the sensation is no longer detected (i.e., a score of 0 on the exemplary scale). In some embodiments, the intensity may increase and decrease multiple times before decreasing to the point that the sensation is no longer detected.

It can be preferable to have a minimum lag between the separate sensations. For example, a lubricant composition with three sensorial agents can be configured for a substantially immediate onset of a first sensation that will increase in intensity and then decrease. The second sensation can onset before the first sensation has completely ceased. The second sensation may then increase in intensity and decrease thereafter. The third sensation also can onset before the second sensation has completely ceased. In this manner the three different sensations are provided sequentially in a temporally spaced manner such that each individual sensation is separately experienced by the user and such that the user is constantly experiencing the provided sensations with no noticeable absence of sensation over the course of use of the lubricant composition. Such experience is provided in relation to the use of two sensorial agents to achieve two sensations, the use of three sensorial agents to achieve three sensations, or the use of four sensorial agents to achieve four sensations. In some embodiments, a first sensorial agent can provide a first sensation and a second sensation that are temporally separated as described herein. A first sensorial agent providing separated first and second sensations can be combined with a second sensorial agent that provides a third sensation (or a third sensation and a fourth sensation).

In certain embodiments, an individual sensation may only be experienced once over the course of use of the lubricant composition. For example, any combination of two, three, four, or all five of warming, cooling, tingling, refreshing, and numbing may be provided sequentially such that the chosen sensation is only provided once. In some embodiments, however, multiple sensorial agents can be used to provide the same sensation separated by a different sensation. For example, warming can be followed by cooling, which can be followed by warming again. Thus, the same sensation can be provided sequentially and temporally spaced while also being separated by the provision of at least one different sensation.

The lubricant composition may also be defined in relation to the total duration of the presence of the sensation provided by a sensorial agent. The duration of a sensation can specifically be measured from the time of onset of the sensation (i.e., the time of first recognition by a user that the sensation is occurring—e.g., a score of 1 on the exemplary intensity scale) to the time that the sensation is no longer perceived (i.e., reaching a score of 0 on the exemplary scale) or is completely masked by a different, higher intensity sensation. In some embodiments, each sensation independently can be imparted to the tissue of application for a duration of about 15 seconds to about 15 minutes, about 30 seconds to about 10 minutes, or about 1 minute to about 5 minutes. In particular embodiments, a sensation can be imparted for a duration of about 15 seconds to about 5 minutes, about 30 seconds to about 4 minutes, or about 1 minute to about 3 minutes.

The sequential and temporally separated nature of the multiple sensations provided by the present lubricant composition can relate directly to the immediate or delayed onset of action of the sensorial agent. It is preferable for the onset of the first sensation provided by the lubricant composition to be substantially immediate—i.e., within 5-10 seconds of contact with the tissue. In further embodiments, the onset of the first sensation occurs within 30 seconds of initial contact to the human skin. Regardless of the time of onset of the first sensation, the onset of the second sensation preferably is delayed until after onset of the first sensation, such as indicated above. Delayed onset of a specific sensation can be achieved in a variety of manners. In some embodiments, the sensorial agent can be chosen based upon the mechanism of action. For example, a self-warming sensorial agent may have a delayed onset until sufficient dilution with moisture has occurred. In further embodiments, the chemical nature of the chosen sensorial agent can define the time of onset as well as the sensation duration. For example, the cooling sensorial agent 2-isopropyl-5-methylcyclohexyl 4-(dimethylamino)-4-oxobutanoate is known to have a time of onset of about 25 seconds after tissue contact and a sensation duration of about 11.25 minutes.

In further embodiments, delayed onset may be provided through formulation techniques. For example, a sensorial agent may be provided in a microencapsulated form such that release of the sensorial agent is controlled. Microcapsules are commercially available, and exemplary types of microcapsule technologies are of the type set forth in Gutcho, Microcapsules and Microencapsulation Techniques (1976); Gutcho, Microcapsules and Other Capsules Advances Since 1975 (1979); Kondo, Microcapsule Processing and Technology (1979); Iwamoto et al., AAPS Pharm. Sci. Tech. 2002 3(3): article 25; U.S. Pat. No. 5,004,595 to Cherukuri et al.; U.S. Pat. No. 5,690,990 to Bonner; U.S. Pat. No. 5,759,599 to Wampler et al.; U.S. Pat. No. 6,039,901 to Soper et al.; U.S. Pat. No. 6,045,835 to Soper et al.; U.S. Pat. No. 6,056,992 to Lew; U.S. Pat. No. 6,106,875 to Soper et al.; U.S. Pat. No. 6,117,455 to Takada et al.; U.S. Pat. No. 6,482,433 to DeRoos et al.; and U.S. Pat. No. 6,929,814 to Bouwmeesters et al.; each of the foregoing being incorporated herein by reference. In some embodiments, menthol, VBE, and eucalyptol are examples of sensorial agents that can be provided in a microencapsulated form.

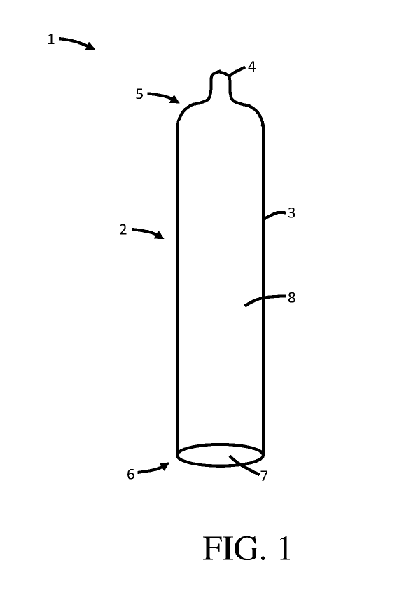

In one or more embodiments, the present disclosure further relates to articles that can include a lubricant as described above. In particular, the article can be a condom that is configured for application to male genitalia. A condom to which the lubricant can be applied can have any shape and/or features as recognized as suitable in the art. Exemplary condom shapes are illustrated in U.S. Pat. No. 7,086,403, the disclosure of which is incorporated herein by reference. As illustrated by the representative embodiment of FIG. 1, a condom 1 according to the present disclosure can comprise a tubular sheath 2 having parallel walls 3 with a nipple 4 at a closed end 5 and having an opposed, open end 6. The nipple 4 may or may not be included at the closed end 5. The walls 3 forming the tubular sheath 2 can include an inner surface 7 and an outer surface 8. In various embodiments, the condom can have a length of about 160 mm to about 220 mm. The nominal length of the condom can be about 180 mm, and the condom can have a lay-flat width of about 52 mm.

The condom may be treated with a lubricant as described herein in a variety of manners. Any of the following application manners may be used.

A lubricant composition as described herein can be applied to all or a portion of the inner surface of the condom wall.

A lubricant composition as described herein can be applied to all or a portion of the outer surface of the condom wall.

A lubricant composition as described herein can be applied to all or a portion of the inner surface of the condom wall, and the same lubricant composition can be applied to all or a portion of the outer surface of the condom wall.

A first lubricant composition as described herein can be applied to all or a portion of the inner surface of the condom wall, and a second, different lubricant composition as described herein can be applied to all or a portion of the outer surface of the condom wail.

A lubricant composition as described herein can be applied to all or a portion of the inner surface of the condom wall, and a known lubricant composition can be applied to all or a portion of the outer surface of the condom wall.

A known lubricant composition can be applied to all or a portion of the inner surface of the condom wall, and a lubricant composition as described herein can be applied to all or a portion of the outer surface of the condom wall.

A lubricant composition as described herein can be applied to only a portion of the inner surface of the condom wall, and the remaining portion of the inner surface of the condom wall can be uncoated or can be coated with a known lubricant.

A lubricant composition as described herein can be applied to only a portion of the outer surface of the condom wall, and the remaining portion of the outer surface of the condom wall can be uncoated or can be coated with a known lubricant.

The lubricant composition can be applied to the condom, or a portion thereof, by any method. For example, U.S. Pat. No. 7,086,403, the disclosure of which is incorporated herein by reference, describes a method whereby a commercial packaging machine provides an indexed motion for moving rolled condoms or whereby motion is timed through the use of rotating cams to activate pumps at appropriate times for ejecting associated lubricants from one or more nozzles. As an exemplary embodiment, the rolled condom can first pass beneath a vertical nozzle for applying a lubricant composition on the exterior surfaces of the condom, and the condom can move over another nozzle for applying another lubricant composition to the interior or inside surfaces of the condom. The condom with the lubricant applied to the desired surface(s) can then be encapsulated between the packaging layers (e.g., foil) and then sealed to form a packaged product as is customary in the art of preparing and packaging condoms.

The amount of the lubricant composition that is applied to the condom can vary. In some embodiments, about a total content of about 0.1 g to about 2.0 g can be applied to one surface of the condom or to both the inner and the outer surfaces of the condom. More particularly, the amount applied can be about 0.4 g to about 1.8 g, about 0.5 g to about 1.5 g, or about 0.6 g to about 1.2 g. The amount of a lubricant composition that is applied to the inner surface and the amount of a lubricant composition that is applied to the outer surface of the condom can be different. When a lubricant composition according to the present disclosure is applied to both of the inner surface and the outer surface, the weight ratio between the inner surface coating and the outer surface coating can be about 10:90 to about 90:10, about 20:80 to about 80:20, or about 40:60 to about 60:40. In some embodiments, the weight ratio between the inner surface coating and the outer surface coating can be about 50:50 to about 10:90, about 40:60 to about 20:80, or about 3070 to about 25:75.

Experimental

Studies were carried out to assess efficacy of various lubricant formulations. Prototypes were first assessed based on a mouth and lip test. If tolerable, the prototype was then assessed for a personal lubricant function in a bedroom panel. Onset of sensations and their duration were profiled as well as their intensity.

Three prototypes were tested—Prototype A, Prototype B, and Prototype C. In all three prototypes, Hotact® VBE and Hotact® VEE were used as warming sensorial agents, and Coolact 20 and WS23 were used as cooling sensorial agents. In Prototype B, TK 1 MM (1% by weight in isopropyl myristate) was included as a warming sensorial agent. In Prototype C, spilanthol was included as a tingling sensorial agent. Concentration ranges for the sensorial agents (based on the total weight of the sensorial agent package) are as follows: Hotact® VBE 5% to 20% by weight; Hotaet® VEE (5% to 10% by weight); TK 1 MM 10% to 20% by weight; Coolact 20 16% to 75% by weight; WS23 15% to 30% by weight; and spilanthol 20% to 30% by weight. The sensorial agent packages were added to a lubricating base of dimethicone and dimethiconol to form the Prototypes. For each Prototype, the sensorial agent package was added to a lubricating base formed of dimethicone and dimethiconol such that the sensorial agent package was 0.1% to 0.7% by weight of the total lubricant composition, and the lubricating base was 99.9% to 99.3% by weight of the total lubricant composition.

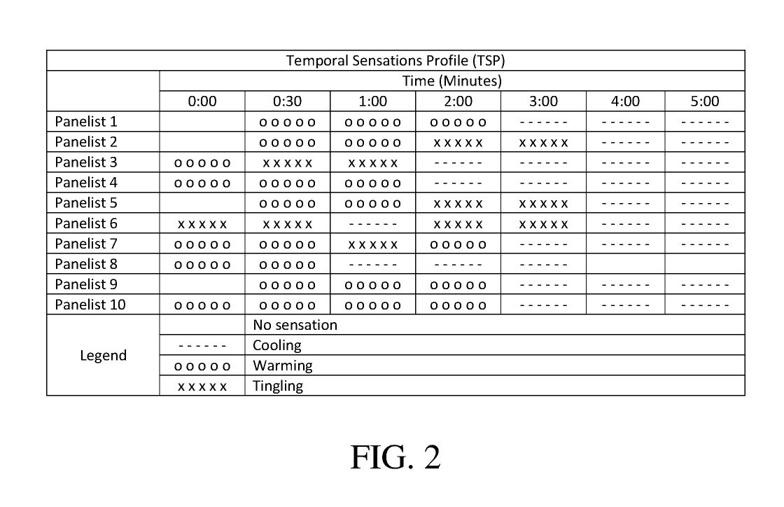

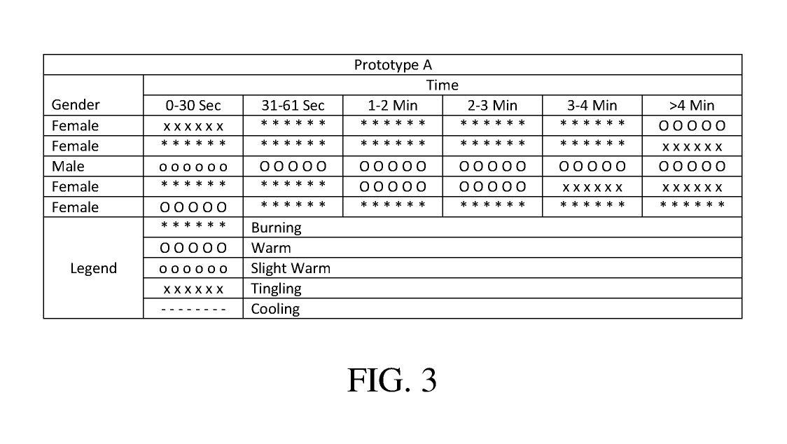

Prototype A was evaluated by 10 panelists in a mouth and lip test. In the test, a cotton swap is dipped into the test solution, and the solution is swabbed across the lip and onto the surface of the gum on the inside of the mouth. This area inside the mouth is composed of a mucosa that is missing the stratum corneum (typically present on other skin as the outmost layer) and is thus especially sensitive. A timer was started at the time of application in the mouth. The panelist then recorded the sensations with their accompanying intensity levels. As seen in FIG. 1, the panelists generally reported a warming sensation followed later by a cooling sensation. Five of the panelists also reported a tingling sensation prior to the cooling sensation. The intensity of the sensations in Prototype were generally reported as being tolerable in the mouth and lip test. Prototype A was then evaluated in a bedroom panel by one male panelist and four female panelists. In the bedroom panel, the subjects were simply instructed to use the test formulation in the same manner as they would otherwise use a personal lubricant and then record their perceptions of the sensations caused by the test formulation. The panelists mainly reported burning or only warming; however, the cooling sensation was generally reported as being absent. See FIG. 2. As illustrated in FIG. 3, the panelists in the bedroom panel for Prototype A reported that the overall intensity was high, and the formulation was not tolerable.

Based upon the results from Prototype A, Prototype B was prepared with reduced concentrations of Hotact® VBE and Hotact® VEE, and TK 1 MM was added. The concentration of Coolact 20 was decreased, and the concentration of WS23 was increased. For Prototype C, the concentrations of Hotact® VBE and Hotact® VEE were both increased relative to Prototype A and Prototype B. The concentration of Coolact 20 was reduced relative to Prototype A and Prototype B, and the concentration of WS23 was increased relative to Prototype A and Prototype B.

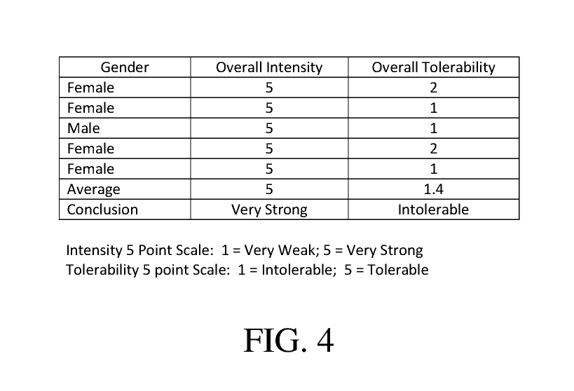

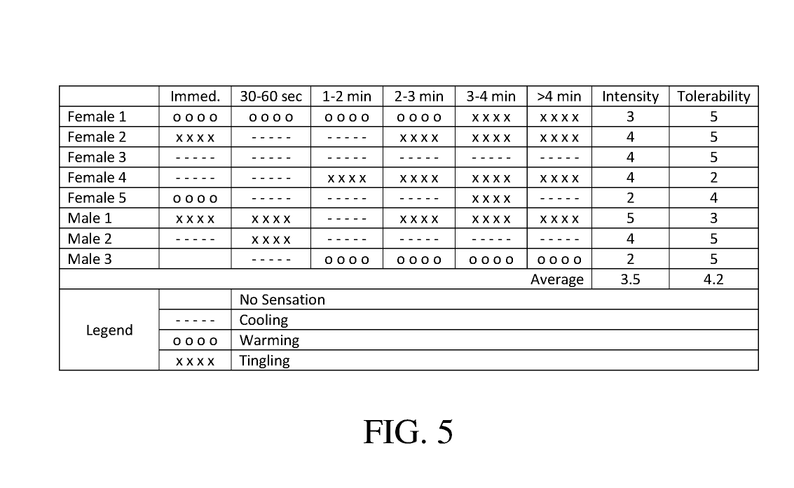

Sensation profiling was assessed for Prototype B and Prototype C using the mouth and lip testing. As seen in FIG. 4, the panelists generally experienced more than two sensations with Prototype B. Panelists also generally reported an acceptable intensity and good tolerability. Similar results were reported with Prototype C, as seen in FIG. 5.

Prototype B and Prototype C were then evaluated in a bedroom panel where a qualitative study with home use testing and focus group input was conducted. Each of Prototype B and Prototype C was used at least twice by 30 heterosexual monogamous couples. Each individual filled out a questionnaire to relate their experience and discuss it at focus group. Prototype B was reported by many of the test subjects to provide multiple sensations, usually warm to warmer to cooling or tingling. They reported that the sensations were highly enjoyable and increased the pleasure of the sexual experience. The consistence of Prototype B was also reported to be “just right” —not sticky or tacky. Further, reapplication of Prototype B was not required during intercourse. Prototype C received mostly neutral to negative reactions from the test subjects. Multiple subjects indicated that the sensations from Prototype C were too intense. Some subjects indicated that the intensity of sensations in Prototype C was so high that the sexual experience was stopped in order to wash off the lubricant. The reported sensations for Prototype C were warming that increased to burning hot (reported as being very uncomfortable) and cool to colder (to the point of numbing).

Based on the qualitative exploratory research, Prototype B was further evaluated in a quantitative study involving 178 heterosexual monogamous couples. Over 90% of panelists reported that Prototype B provided changing sensations during use, and over 80% of panelists characterized Prototype B as being a lubricant that provided a series of sensations over time. 100% of panelists reported Prototype B as providing enjoyable sensations.

As described above, testing indicated that Prototype B provided the most desired results. Based on such results, preferred sensorial agent packages for lubricant compositions can include about 10% to about 25% by weight of warming sensorial agents and about 75% to about 90% by weight of cooling sensorial agents based on the total weight of the sensorial agent package. In particular embodiments, preferred sensorial agent packages for lubricant compositions can include the following (percentages being based on the total weight of all sensorial agents used in the lubricant composition): Hotact® VBE (1% to 8% by weight); Hotact® VEE (1% to 8% by weight); TK 1 MM (10% to 20% by weight); Coolact 20 (55% to 75% by weight); and WS23 (5% to 35% by weight).

Many modifications and other embodiments of the inventions set forth herein will come to mind to one skilled in the art to which these inventions pertain having the benefit of the teachings presented in the foregoing descriptions. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. A lubricant comprising:
a lubricating base;
a first sensorial agent effective to impart a first sensation to contacted human tissue; and
a second sensorial agent effective to impart a second sensation to the contacted human tissue, the second sensation being different from the first sensation;
wherein onset of the first sensation and onset of the second sensation are sequential and are temporally separated.

2. The lubricant of claim 1, further comprising a third sensorial agent effective to impart a third sensation to the contacted human tissue, the third sensation being different from the first sensation and the second sensation, wherein onset of the third sensation and onset of the second sensation are sequential and temporally separated.

3. The lubricant of claim 1, wherein intensity of one or both of the first sensation and the second sensation varies over the time of contact with the human tissue.

4. The lubricant of claim 3, wherein intensity of one or both of the first sensation and the second sensation gradually increases to a maximum intensity and then gradually decreases.

5. The lubricant of claim 1, wherein the onset of the first sensation and onset of the second sensation are temporally separated by a time of about 5 seconds or greater.

6. The lubricant of claim 5, wherein the onset of the first sensation and onset of the second sensation are temporally separated by a time of about 5 seconds to about 5 minutes.

7. The lubricant of claim 2, wherein the onset of the second sensation and the onset of the third sensation are temporally separated by a time of about 5 seconds or greater.

8. The lubricant of claim 2, wherein the onset of the second sensation and onset of the third sensation are temporally separated by a time of about 5 seconds to about 5 minutes.

9. The lubricant of claim 1, wherein the first sensation and the second sensation are each independently imparted for a duration of about 30 seconds to about 15 minutes.

10. The lubricant of claim 2, wherein the third sensation is independently imparted for a duration of about 30 seconds to about 15 minutes.

11. The lubricant of claim 1, wherein the first sensation substantially ceases before the onset of the second sensation.

12. The lubricant of claim 2, wherein the second sensation substantially ceases before the onset of the third sensation.

13. The lubricant of claim 1, wherein the imparting of the first sensation overlaps with the imparting of the second sensation by a time of no more than 3 minutes.

14. The lubricant of claim 2, wherein the imparting of the second sensation overlaps with the imparting of the third sensation by a time of no more than 3 minutes.

15. The lubricant of claim 1, wherein the first sensation and the second sensation are independently selected from the group consisting of warming, cooling, tingling, numbing, and refreshing.

16. The lubricant of claim 2, wherein the third sensation is selected from the group consisting of warming, cooling, tingling, numbing, and refreshing.

17. The lubricant of claim 1, wherein one of the first sensorial agent and the second sensorial agent is a warming sensorial agent, and the other of the first sensorial agent and the second sensorial agent is a cooling sensorial agent.

18. The lubricant of claim 2, wherein one of the first sensorial agent, the second sensorial agent, and the third sensorial agent is a warming sensorial agent, and another of the first sensorial agent, the second sensorial agent, and the third sensorial agent is a cooling sensorial agent, and the remaining of the first sensorial agent, the second sensorial agent, and the third sensorial agent is a tingling sensorial agent.

19. The lubricant of claim 1, wherein the onset of the first sensation occurs within 30 seconds of initial contact to the human skin, and the onset of the second sensation is delayed for at least 1 minute after initial contact to the human skin.

20. A method of providing lubrication and varying sensations to human tissue, the method comprising applying a lubricant to the human tissue, wherein the lubricant comprises:
a lubricating base;
a first sensorial agent effective to impart a first sensation to contacted human tissue; and
a second sensorial agent effective to impart a second sensation to the contacted human tissue, the second sensation being different from the first sensation;
wherein onset of the first sensation and onset of the second sensation are sequential and are temporally separated.

21. A packaged condom comprising:
a male genital engaging tubular sheath having an inner surface and an outer surface; and
a lubricant applied to at least a portion of one or both of the inner surface and the outer surface of the tubular sheath, the lubricant comprising:
a lubricating base;
a first sensorial agent effective to impart a first sensation to contacted human tissue; and
a second sensorial agent effective to impart a second sensation to the contacted human tissue, the second sensation being different from the first sensation;
wherein onset of the first sensation and onset of the second sensation are sequential and are temporally separated.
